(19) United States
(12) Patent Application Publication
(10) Pub. No.: US 2021/0391073 A1
(43) Pub. Date: Dec. 16, 2021

(54) A SYSTEM AND METHOD TO PROCESS WAVEFORM DATA IN MEDICAL DEVICES

(71) Applicant: KONINKLIJKE PHILIPS N.V., EINDHOVEN (NL)

(72) Inventors: MAHMOUDREZA SHARIFI, NEW ROCHELLE, NY (US); IKARO GARCIA ARAUJO DA SILVA, WATERTOWN, MA (US); CORNELIS CONRADUS ADRIANUS MARIA VAN ZON, EVERETT, MA (US)

(21) Appl. No.: 17/289,100

(22) PCT Filed: Nov. 12, 2019
(86) PCT No.: PCT/EP2019/080910

§ 371 Date: Apr. 27, 2021

Related U.S. Application Data
(60) Provisional application No. 62/758,839, filed on Nov. 12, 2018.

(51) Int. Cl. G16H 40/67; G16H 50/30; G16H 10/60

(52) U.S. Cl. CPC G16H 40/67; G16H 10/60; G16H 50/30

(57) ABSTRACT

Various embodiments relate to a programmable interface configured to connect a medical device to a main platform, including: a medical device interface configured to connect to a medical device, receive waveform data from the medical device, and to send medical device commands to the medical device; a main platform interface configured to connect to a main platform, receive platform interface commands from the main platform, and transmit waveform data from the medical device to the main platform based upon the platform interface commands; a buffer memory configured to store waveform data generated by the medical device; and a waveform processing and control unit configured to process the waveform data generated by the medical device stored in the buffer memory according to the platform interface commands.

### TECHNICAL FIELD

This disclosure relates generally to providing an architectural solution to control an analytics platform for a network of medical devices. This platform enables third party software developers' easy access to settings, alarms, numeric, and real-time waveforms from several medical devices synchronously. It also provides access to the change in the setting of such devices in a service-oriented architecture.

### BACKGROUND

Medical devices and instruments generate measurement data from the patients when they are used by medical personnel. Medical personnel use increasing numbers of medical devices such as medical devices with sensing technology, wearable medical devices and therefore the amount of measurement data that is generated by these medical devices is increasing.

This measurement data is a valuable source of patient health condition information and if the measurement data can be processed, it may have an impact on the diagnosis, prognosis and quality of healthcare delivery to a patient.

Currently, the use of this measurement data is under-utilized because of the difficulty of managing the measurement data in real time. One type of measurement data that is ignored by medical personnel and not even captured in the medical record is the waveform data. For example, a ventilator may generate pressure and flow waveform data that may be an indicator of the patient's lung mechanics and respiratory muscle activity. This type of measurement data in waveforms is displayed in real-time on a patient monitor and medical personnel, such as a respiratory therapist may capture some of the information by looking at the monitor. However, in the absence of medical personnel, much of this measurement data is unnoticed.

Waveform data is not normally recorded, but even if it is recorded, it may be time consuming to go over the past history of the patient data to detect potential issues or indicators in the past few hours. For example, by using measurement data, improved outcomes may be obtained through a detailed analysis of waveform data in the intensive care unit (“ICU”) including prediction of mortality, hypotensive episodes and reduction of false alarms.

### SUMMARY

A brief summary of various embodiments is presented below. Embodiments address a system and method to process waveform data in medical devices.

A brief summary of various example embodiments is presented. Some simplifications and omissions may be made in the following summary, which is intended to highlight and introduce some aspects of the various example embodiments, but not to limit the scope of the invention.

Detailed descriptions of example embodiments adequate to allow those of ordinary skill in the art to make and use the inventive concepts will follow in later sections.

Various embodiments relate to a programmable interface configured to connect a medical device to a main platform, including: a medical device interface configured to connect to a medical device, receive waveform data from the medical device, and to send medical device commands to the medical device; a main platform interface configured to connect to a main platform, receive platform interface commands from the main platform, and transmit waveform data from the medical device to the main platform based upon the platform interface commands; a buffer memory configured to store waveform data generated by the medical device; and a waveform processing and control unit configured to process the waveform data generated by the medical device stored in the buffer memory according to the platform interface commands.

Various embodiments are described, wherein the platform interface commands received from the main platform are configured to remotely program the programmable interface to receive waveform data based on a defined logic.

Various embodiments are described, wherein the medical device connects to the main platform through plug-and-play.

Various embodiments are described, wherein the programmable interface is configured to store a serial number and identification the medical device to allow the main platform to communicate with the medical device.

Various embodiments are described, wherein the programmable interface is configured to continuously receive the waveform data from the medical device and store a last set of waveform data in the buffer memory in a raw form.

Various embodiments are described, wherein the programmable interface is configured to transmit information of services that the medical device can perform to the main platform.

Various embodiments are described, wherein the programmable interface is configured to change settings in the medical device based on programmable interface commands from the main platform.

Various embodiments are described, wherein the waveform processing and control unit is configured to perform resampling of waveform data.

Various embodiments are described, wherein the waveform processing and control unit is configured to perform sanity checks on the waveform data.

Various embodiments are described, wherein the waveform processing and control unit is configured to extract single variable features from the medical device waveform data and send the waveform data to the main platform at a requested frequency.

Various embodiments are described, wherein the programmable interface is configured to synchronize time with the medical device.

Further various embodiments relate to a method of connecting a medical device to a main platform by a programmable interface, including: receiving platform interface commands from the main platform, receiving waveform data from the medical device, sending medical device commands to the medical device; storing waveform data generated by the medical device in a buffer memory; processing the waveform data generated by the medical device stored in the buffer memory according to the platform interface commands; and transmitting waveform data from the medical device to the main platform based upon the platform interface commands.

Various embodiments are described, wherein the platform interface commands received from the main platform are configured to remotely program the programmable interface to receive a waveform based on a defined logic.

Various embodiments are described, wherein the medical device connects to the main platform through plug-and-play.

Various embodiments are described, further including storing a serial number and identification the medical device to allow the main platform to communicate with the medical device.

Various embodiments are described, further including continuously receiving the waveform data from the medical device and storing a last set of waveform data in the buffer memory in a raw form.

Various embodiments are described, further including transmitting information of services that the medical device can perform to the main platform.

Various embodiments are described, further including changing settings in the medical device based on programmable interface commands from the main platform.

Various embodiments are described, further including performing resampling of waveform data.

Various embodiments are described, further including performing sanity checks on the waveform data.

Various embodiments are described, further including extracting single variable features from the medical device waveform data and sending the waveform data to the main platform at a requested frequency.

Various embodiments are described, further including synchronizing time with the medical device.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate example embodiments of concepts found in the claims and explain various principles and advantages of those embodiments.

These and other more detailed and specific features are more fully disclosed in the following specification, reference being had to the accompanying drawings, in which:

FIG. 1 illustrates a block diagram of the system to process waveform data in medical devices of the current embodiment; and

FIG. 2 illustrates a block diagram of the components of the programmable interface of the current embodiment.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments. Descriptors such as “first,” “second,” “third,” etc., are not meant to limit the order of elements discussed, are used to distinguish one element from the next, and are generally interchangeable.

In the current embodiment, in order to run online algorithms on measurement data, for example, waveform data, the raw signals may need to be sent to the analytics platform continually and with minimum latency. While this is may not be an issue for a select measurement data, as the number of measurements increases, it may become challenging to ensure the raw signals are being received continually and with minimum latency because these raw signals may be transformed over network connections and there may be a network bandwidth limitation for receiving all of these raw signals together and the system memory requirements may not permit several high frequency incoming signals at the same time and finally, processing all of this measurement data may consume processing power of the analytics platform.

However, analysis of measurement data, including the waveform data may not need to be analyzed all the time. For example, there may be periods of time that there is no change in the patient condition and therefore, processing the waveform data may not add any new information. Therefore, the current embodiment is directed towards a smart algorithm to sample and send the measurement data into an analytics platform only when it is needed which may improve the performance of the analytics platform as well as reduce network traffic.

The current embodiment is directed towards allowing third-party developers to either select from a list of pre-designed period triggering algorithms or develop their own logic for smart sampling of the data. There may be three different levels of smart sampling depending on the role of the application and on the sampling logic.

The first level allows the third-party developer to only select from a set of the existing sampling logic. In this level, there may be libraries of different sampling logics that are previously programmed into the platform. For example, one logic would be to send five seconds of an electrocardiogram (“ECG”) signal when there is change in the infusion rate or send 10 seconds of capnography waveform every five minutes.

The second level allows the third-party developer to define parameters of the selection criteria. In this level, the third-party developer may have freedom to may use the parameters of the logic. For example, send m seconds of data every n minutes and the m and n are defined by the third-party developer. Another example would be to send t seconds of data when heart rate is more that x and t and x are defined by the user.

The third level allows the application programs to select logic. In this level, the logic is defined by the application. This is in case the third-party developer does not find its intended logic among the list of predetermined logics.

The current embodiment is directed towards the three levels of sampling logic, however, in order to implement these three levels of sampling logic, an implementation architecture is required.

This architecture requires memory and processing power at the device interface, where each medical device is connected to a dedicated computer that manages the real-time data collected from that medical device and communicates with the analytical platforms.

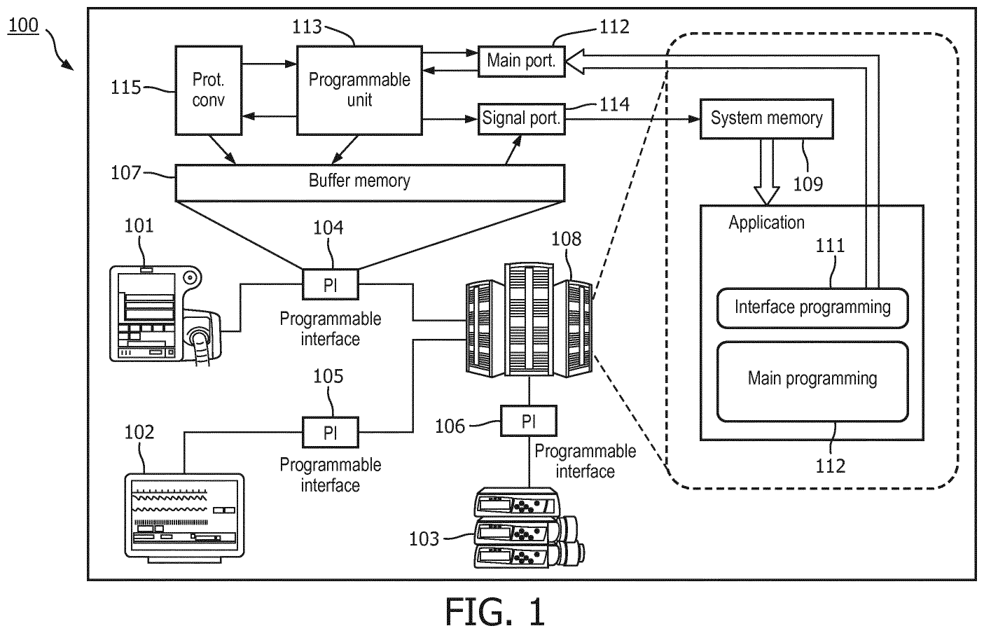

FIG. 1 illustrates a block diagram of the system platform 100 to process waveform data in medical devices of the current embodiment.

The system platform 100 of the high-level architecture of the medical device platform illustrates each medical device 101, 102 and 103 being connected to dedicated programmable interfaces (“PIs”) 104, 105 and 106.

The function of the PIs 104, 105 and 106 is to facilitate the integration of the medical devices 101, 102 and 103 into the network 108. The PIs 104, 105 and 106 communicate with the medical devices 101, 102 and 103 with an interface and continuously read all the data generated from the medical device 101, 102 and 103 and stores it to the buffer memory 107.

Once the medical devices 101, 102 and 103 are connected to the network 108 through its PIs 104, 105 and 106, the medical devices 101, 102 and 103 will be automatically registered into the system as a new medical device with all of medical device's properties and capabilities as a new service. Therefore, the PIs 104, 105 and 106 declare and enlist the medical device 101, 102 and 103 services into the main platform 110.

The network 108 includes a system memory 109 and an application 110 which includes interface programming 111 and main programming 112.

In an alternate embodiment, the medical devices 101, 102 and 103 that support plug and play standards may be managed directly by the central station.

The main platform 110 may be connected to multiple medical devices 101, 102 and 103 through their PIs 104, 105 and 103 and may be able to monitor the operation of these medical devices 101, 102 and 103 remotely. The main platform 110 may provide application programming interfaces (“APIs”) 111 for external developers to access the network 108 of medical devices 101, 102 and 103 and perform real-time analytic tasks. It may also be capable of remotely programming each API 111 to receive a certain waveform based on a specific logic.

The API 111 is connected to the main port 112 which is connected to a programmable unit 113 and a signal port 114. The main port 112 receives instructions from the API 111 that define the data to be collected and how it is to be collected. The signal port 114 is connected to the system memory 109 of the main platform 110 and sends collected waveform data to the system memory 109. The protocol conversation block. 115 is connected to the buffer memory 107 and the programmable unit 113. The protocol conversion block unpacks the device data using the device specific communication protocol The programmable unit 113 is connected to the protocol conversion block 115, the buffer memory 107, the main port 112 and the signal port 114. The programmable unit 113 controls the overall operation of the programmable interfaces 104, 105 and 106.

The PIs 104, 105 and 106 perform the following tasks: 1) connecting to the medical devices 101, 102 and 103 with proprietary communication protocols of the medical devices 101, 102 and 103; 2) storing the unique serial number and ID of the medical device 101, 102 and 103 used by the platform 100 for addressing the medical device 101, 102 and 103; 3) continuously receiving the data and waveforms generated by the medical devices 101, 102, and 103 and keeping the last T seconds of the medical devices 101, 102 and 103 data into buffer memory 107 in raw form; 4) connecting to the main platform 110 as plug and play system; 5) registering all services that the medical devices 101, 102 and 103 may provide into the system platform's 100 service registry; 6) receiving commands from the main platform 113; 7) performing changes in the medical devices 101, 102 and 103 settings based on the commands from the main platform 110; 8) sending the waveform data from buffer memory 107 to the system platform 100 upon activation of certain logic; 9) performing resampling of the waveform data; 10) performing a sanity check of the waveform data; 11) extracting single variable features from the medical devices 101, 102 and 103 waveform and sending it to the system platform 100 at the requested frequency; 12) extracting features from multiple waveforms by networking to other PIs 104, 105 and 106; and 13) synchronizing the time between different medical devices, because all PIs 104, 105 and 106 use a single time server.

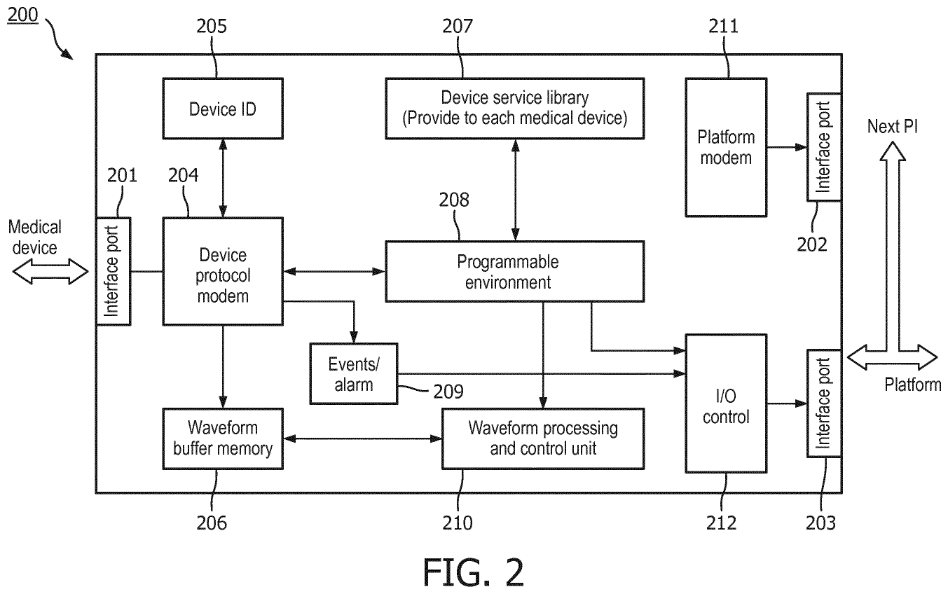

FIG. 2 illustrates a block diagram of the components of the programmable interface 200 of the current embodiment.

The device interface port 201 is a communication port dedicated to all communications between the medical device and the interface and depending of the type of the device, the device interface port 201 may be either an Ethernet port or an RS232 port and the device interface port 201 may support both commands and data communications.

The network interface port 202 is an TCP/IP port for receiving and sending the commands and programs from the main platform 110. The network interface port 202 and the steaming port 203 may share the same physical Ethernet port.

The streaming port 203 is a port that may be used to stream the waveforms collected from the medical device into the network. The streaming port 203 may use the same Ethernet connection as the network interface port 202.

The device protocol modem 204 coverts the data and commands into the medical device specific communication protocol and vice versa. The device ID 205 is a unique identifier that is dedicated to each single medical device and is used to locate and find the medical device in the network and address it. This device ID may be stored in a general device memory or in a specific dedicated memory.

The waveform buffer memory 206 is a memory for keeping the latest waveform data. The last T seconds of waveform data is stored in the waveform buffer memory 206 even if it is not being used by any application. The waveform buffer memory 206 is continuously updated and the unused data after T seconds is discarded.

The device service library 207 is a library of all services that the medical device can provide. It is pre-programmed for each device and will remain constant. The device service library 207 may enlist the device services into the service repository of the system platform. For example, for a mechanical ventilator interface, some of the services will change the pressure support level, silent the alarm and provide the patients respiratory rate, etc.

The programmable environment 208 is an environment which provides the capability to program the interface and customize it depending on the need of the application. For example, the programmable environment 208 may configure the interface to send the waveform data depending on certain events. The programmable environment 208 may include a processor or microcontroller running program instructions that allows for the overall control of the programmable interface.

The event manager 209 may receive the event in the device and send it to the signal I/O control.

The waveform processing unit 210 performs the primary signal processing on the data. For example, the waveform processing unit 210 may filter the data or remove artifacts from the waveform data in the waveform processing unit 210.

The platform modem 211 may communicate with the central platform. The communication between the platform and the device may be in the form of JASON or TCP/IP, but other protocols may also be used. Further, the platform modem 211 may perform network security functions to protect the data and programmable interface 200 from attack.

The I/O control 212, according to the logic used in the programming, may publish or store the waveform data into the main platform.

It should be apparent from the foregoing description that various exemplary embodiments of the invention may be implemented in hardware. Furthermore, various exemplary embodiments may be implemented as instructions stored on a non-transitory machine-readable storage medium, such as a volatile or non-volatile memory, which may be read and executed by at least one processor to perform the operations described in detail herein. A non-transitory machine-readable storage medium may include any mechanism for storing information in a form readable by a machine, such as a personal or laptop computer, a server, or other computing device. Thus, a non-transitory machine-readable storage medium may include read-only memory (ROM), random-access memory (RAM), magnetic disk storage media, optical storage media, flash-memory devices, and similar storage media and excludes transitory signals.

It should be appreciated by those skilled in the art that any blocks and block diagrams herein represent conceptual views of illustrative circuitry embodying the principles of the invention. Implementation of particular blocks can vary while they can be implemented in the hardware or software domain without limiting the scope of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Accordingly, it is to be understood that the above description is intended to be illustrative and not restrictive. Many embodiments and applications other than the examples provided would be apparent upon reading the above description. The scope should be determined, not with reference to the above description or Abstract below, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

What is claimed is:

1. A programmable interface configured to connect a medical device to a main platform, comprising:
a medical device interface configured to connect to a medical device, receive waveform data from the medical device, and to send medical device commands to the medical device;
a main platform interface configured to connect to a main platform, receive platform interface commands from the main platform, and transmit waveform data from the medical device to the main platform based upon the platform interface commands;
a buffer memory configured to store waveform data generated by the medical device; and
a waveform processing and control unit configured to process the waveform data generated by the medical device stored in the buffer memory according to the platform interface commands.

2. The programmable interface of claim 1, wherein the platform interface commands received from the main platform are configured to remotely program the programmable interface to receive waveform data based on a defined logic.

3. The programmable interface of claim 1, wherein the medical device connects to the main platform through plug-and-play.

4. The programmable interface of claim 1, wherein the programmable interface is configured to store a serial number and identification the medical device to allow the main platform to communicate with the medical device.

5. The programmable interface of claim 1, wherein the programmable interface is configured to continuously receive the waveform data from the medical device and store a last set of waveform data in the buffer memory in a raw form.

6. The programmable interface of claim 1, wherein the programmable interface is configured to transmit information of services that the medical device can perform to the main platform.

7. The programmable interface of claim 1, wherein the programmable interface is configured to change settings in the medical device based on programmable interface commands from the main platform.

8. The programmable interface of claim 1, wherein the waveform processing and control unit is configured to perform resampling of waveform data.

9. The programmable interface of claim 1, wherein the waveform processing and control unit is configured to perform sanity checks on the waveform data.

10. The programmable interface of claim 1, wherein the waveform processing and control unit is configured to extract single variable features from the medical device waveform data and send the waveform data to the main platform at a requested frequency.

11. The programmable interface of claim 1, wherein the programmable interface is configured to synchronize time with the medical device.

12. A method of connecting a medical device to a main platform by a programmable interface, comprising:
receiving platform interface commands from the main platform,
receiving waveform data from the medical device,
sending medical device commands to the medical device;
storing waveform data generated by the medical device in a buffer memory;
processing the waveform data generated by the medical device stored in the buffer memory according to the platform interface commands; and
transmitting waveform data from the medical device to the main platform based upon the platform interface commands.

13. The method of claim 12, wherein the platform interface commands received from the main platform are configured to remotely program the programmable interface to receive a waveform based on a defined logic.

14. The method of claim 12, wherein the medical device connects to the main platform through plug-and-play.

15. The method of claim 12, further comprising storing a serial number and identification the medical device to allow the main platform to communicate with the medical device.

16. The method of claim 12, further comprising continuously receiving the waveform data from the medical device and storing a last set of waveform data in the buffer memory in a raw form.

17. The method of claim 12, further comprising transmitting information of services that the medical device can perform to the main platform.

18. The method of claim 12, further comprising changing settings in the medical device based on programmable interface commands from the main platform.

19. The method of claim 12, further comprising performing resampling of waveform data.

20. The method of claim 12, further comprising performing sanity checks on the waveform data.

21. The method of claim 12, further comprising extracting single variable features from the medical device waveform data and sending the waveform data to the main platform at a requested frequency.

22. The method of claim 12, further comprising synchronizing time with the medical device.
